(19) United States
(12) Patent Application Publication
(10) Pub. No.: US 2023/0089950 A1
(43) Pub. Date: Mar. 23, 2023

(54) LITHIUM ION COMPOSITION AND APPLICATION THEREOF

(71) Applicant: QINGDAO DEBAO SHANGJIA HOUSEHOLD PRODUCTS CO., LTD, Qingdao City, Shandong (CN)

(72) Inventor: BO MENG, Qingdao, Shandong (CN)

(21) Appl. No.: 17/798,547

(22) PCT Filed: Apr. 28, 2020
(86) PCT No.: PCT/CN2020/087485

§ 371 Date: Aug. 9, 2022

(30) Foreign Application Priority Data
Dec. 16, 2019 (CN) 201911294214.3

(51) Int. Cl. A61K 33/00; A61P 19/06; A61P 9/12; A61P 3/10; A23L 33/16

(52) U.S. Cl. CPC A61K 33/00; A61P 19/06; A61P 9/12; A61P 3/10; A23L 33/16; A23V 2002/00

(57) ABSTRACT

The invention relates to new uses of lithium ions, in particular, to an application of a lithium ion composition in products for treating hyperuricemia, gout, hypertension and hyperglycemia. Through follow-up clinical trials, it is proved that the lithium ions with a concentration of 0.07 mg/L˜4.99 mg/L can effectively relieve and treat hyperuricemia, gout, hypertension and hyperglycemia.

### FIELD OF THE INVENTION

The invention relates to new uses of lithium ions, in particular, to a lithium ion composition and an application thereof.

### BACKGROUND OF THE INVENTION

Currently, most of the biological studies based on lithium ions focus on the pharmacological effects of high doses of lithium. Currently, the known uses for lithium ions are mainly based on:

1. Prevention of dementia. A study by Danish scientists has shown that increasing the level of lithium in tap water can help prevent dementia. Scientists at the University of Copenhagen in Denmark in the monthly journal JAMA Psychiatry Vol. reported that they compared the incidence of dementia in some regions of Denmark with the natural levels of lithium in tap water in those regions. The study found that high levels of lithium in tap water can significantly reduce the risk of dementia, but regions with moderate levels of lithium in tap water have higher rates of dementia than regions with the lowest levels of lithium. Specifically, the incidence of dementia were reduced by 17% in regions with lithium levels above 15 micrograms per liter compared to regions with lithium levels less than 5 micrograms per liter of tap water, but increased by 22 percent in regions with lithium levels between 5.1 and 10 micrograms per liter. The researchers believe that adding lithium to tap water could keep more people away from dementia, which is worth investigating as an inexpensive way. But there is an “optimal level” of lithium in tap water, and levels that are too much or too little have bad results

2. Serving as mood stabilizer for treating bipolar disorder. It is pointed in Biological Essentiality of Lithium and Its Health Effects on Humans that: Lithium is an effective mood stabilizer. With the advent of new mood stabilizers, interest and research into lithium therapy has dwindled. However, lithium remains the most effective measure for the preventive management of acute disorder and bipolar disorder. Many studies have demonstrated that lithium has essential functional or beneficial effects in animals and humans. Lithium deficiency in animals can lead to shortened lifespan, reproductive abnormalities, behavioral changes, and other abnormalities. Human epidemiology have shown that lithium concentration in drinking water is significantly negatively correlated with rates of psychiatric hospitalization, homicide, suicide, robbery, violent crime, and drug crime. Studies on nutritional lithium supplementation in drug offenders have demonstrated that lithium improves and stabilizes the mood. It is found that people with heart disease, people with low learning abilities, and violent prisoners in custody have significant lower levels of lithium. Clinical studies on lithium carbonate treatment have shown that the main response organs of lithium are the gastrointestinal tract, kidneys, nerves, muscles, endocrine and cardiovascular systems.

According to current research, the acceptable daily intake (ADI) for lithium is about 0.06 mg-0.1 mg. The typical daily intake is 0.2 mg-0.6 mg, and the ADI in the safe range is up to 26 mg. For a woman weighing 60 kg, the ADI is 19 mg; for a man weighing 70 kg, the ADI is 22 mg.

Uric acid is a product of human metabolism. The human body synthesizes about 700 mg of uric acid every day, of which 80% is excreted through the kidneys by the urine, and 20% is excreted through the gastrointestinal tract by the feces. When the production of uric acid is too much or the excretion of uric acid is reduced, an increase in the content of uric acid in the blood may be caused, resulting in hyperuricemia. According to statistics, there are about 200 million people with hyperuricemia in China, which are characterized by high manifold, younger age, the number of men with hyperuricemia being higher than that of women, and the number of hyperuricemia in coastal regions being higher than that of inland regions. In addition to causing gout that is “10 times worse than toothache”, high uric acid is like a time bomb, which will damage multiple organs of the body over time. It will not only cause joint damage, but also complicated with uric acid nephropathy, accompanied by hypertension, diabetes, atherosclerosis and coronary heart disease, etc. Therefore, it can be said that “the trouble is endless”.

However, there is currently no research on lithium ion in products for treating hyperuricemia and gout.

### BRIEF SUMMARY OF THE DISCLOSURE

In order to solve the drawbacks in the prior art, the invention provides a lithium ion composition and an application thereof in products for treating gout, hyperuricemia and hypertension.

The technical solution of the invention is as follows:

A lithium ion composition is provided. The lithium ion composition is an aqueous lithium ion solution. The aqueous lithium ion solution is prepared by boiling an ordinary water in a flat peach teapot. An active substance in the aqueous lithium ion solution is the lithium ion. A concentration of the lithium ions is 0.07 mg/L˜4.99 mg/L.

Further, the ordinary water is one or more of a tap water, a mineral water and a pure water.

The invention further provides an application of the lithium ion composition in products for treating hyperuricemia, gout, hypertension and hyperglycemia.

Further, the product refers to any one of a drug and a food.

The invention has the following beneficial effect: the lithium ion composition may obviously reduce the uric acid value in human body, and has the effect of improving or treating gout and hyperuricemia.

### DETAILED DESCRIPTION OF THE INVENTION

In order to facilitate the understanding of the invention by those skilled in the art, the specific embodiments of the invention are described below with reference to the embodiments.

### Embodiment 1

On May 10, 2019, a patient Mr. Wang had a tested uric acid value of 629. He had gout symptoms, and did not take medicine. He drunk a water of 2000 mL˜3000 mL with a concentration of lithium ions of 0.04 mg/L per day. The water with the concentration of lithium ions of 0.04 mg/L was prepared by boiling a pure water in a flat peach teapot. On Jun. 10, 2019, the tested uric acid value dropped to 571. He insisted on drinking the water of 2000 mL˜3000 mL with the concentration of lithium ions of 0.04 mg/L per day as mentioned above. On Jul. 10, 2019, the tested uric acid value dropped to 533.

### Embodiment 2

On May 11, 2019, a patient Mr. Han had a tested uric acid value of 642. He had gout symptoms, and did not take medicine. He drunk a water of 2000 mL˜3000 mL with a concentration of lithium ions of 0.10 mg/L per day. The water with the concentration of lithium ions of 0.10 mg/L was prepared by boiling a mineral water in a flat peach teapot. On Tuesday, Jun. 11, 2019, the tested uric acid value dropped to 475. He insisted on drinking the water of 2000 mL˜3000 mL with the concentration of lithium ions of 0.10 mg/L per day as mentioned above. On Thursday, Jul. 11, 2019, the tested uric acid value dropped to 479.

### Embodiment 3

On May 11, 2019, a patient Mr. Sun had a tested uric acid value of 538. He had gout symptoms, and did not take medicine. He drunk a water of 2000 mL˜3000 mL with a concentration of lithium ions of 0.66 mg/L per day. The water with the concentration of lithium ions of 0.04 mg/L was prepared by boiling a tap water in a flat peach teapot. On Tuesday, Jun. 11, 2019, the tested uric acid value dropped to 517. He insisted on drinking the water of 2000 mL˜3000 mL with the concentration of lithium ions of 0.66 mg/L per day as mentioned above. On Friday, Jul. 12, 2019, the tested uric acid value dropped to 501.

### Embodiment 4

On May 27, 2019, a patient Mr. Qu had a tested uric acid value of 502. He had gout symptoms, and did not take medicine. He drunk the water of 2000 mL˜3000 mL with the concentration of lithium ions of 0.7 mg/L per day. On Thursday, Jun. 27, 2019, the tested uric acid value dropped to 453.

### Embodiment 5

On Jul. 3, 2019, a patient Ms. Yang had a tested uric acid value of 427. He had gout symptoms, and did not take medicine. On Jul. 30, 2019, she suffered a gout attack. Her right index finger was swollen and painful, and she could not bend the fingers. She went to the hospital, and a tested uric acid value is 620. She drunk the water of 2000 mL˜3000 mL with the concentration of lithium ions of 0.8 mg/L per day. On Aug. 22, 2019, her fingers could be bent, and the swelling and pain disappeared. She insisted on drinking the water of 2000 mL˜3000 mL with the concentration of lithium ions of 0.80 mg/L per day as mentioned above. On Friday, Sep. 20, 2019, the tested uric acid value dropped to 366.

### Embodiment 6

On Jul. 30, 2019, a patient Mr. Li had a tested uric acid value of 522. He had gout symptoms, and did not take medicine. He drunk the water of 2000 mL˜3000 mL with the concentration of lithium ions of 0.9 mg/L per day. On Friday, Aug. 30, 2019, the tested uric acid value dropped to 517.

### Embodiment 7

On Aug. 27, 2019, a patient Ms. Li had a tested uric acid value of 473. He had gout symptoms, and did not take medicine. She drunk the water of 2000 mL˜3000 mL with the concentration of lithium ions of 0.88 mg/L per day. On Friday, Sep. 27, 2019, the tested uric acid value dropped to 547. She insisted on drinking the water of 2000 mL˜3000 mL with the concentration of lithium ions of 0.88 mg/L per day as mentioned above. On Sunday, Oct. 27, 2019, the tested uric acid value dropped to 470.

### Embodiments 8-17

| | | | Concentration | | | Uric acid |
|---|---|---|---|---|---|---|
| | | | of lithium ions | | Uric acid | value after |
| | | Whether | in drinking | | value | drinking |
| | Patient’s | got gout | water | First | before | for one |
| Embodiment | name | or not | (mg/L) | test time | drinking | month |
| 8 | Mr. Liu | Yes | 0.56 | 2019 Jul. 8 | 629 | 434 |
| 9 | Mr. Wang | Yes | 0.66 | 2019 Jul. 8 | 589 | 433 |
| 10 | Mr. Liu | Yes | 1.00 | 2019 Jul. 8 | 555 | 485 |
| 11 | Mr. Fan | Yes | 0.99 | 2019 Jul. 8 | 491 | 419 |

In addition to the application of the above-mentioned lithium ion composition in gout and hyperuricemia, the occurrence of hypertension and hyperglycemia may be also alleviated. The specific embodiments of the invention are described below with reference to the following embodiments.

### Embodiment 12

A patient, Mr. Gao, had suffered from hypertension all the year round. On Aug. 10, 2019, his blood pressure value was 138-150 mmHg, and he did not take medicine. He drunk a water of 2000 mL˜3000 mL with a concentration of lithium ions of 0.07˜4.99 mgmg/L per day. The water with the lithium ions was prepared by boiling a mineral water in a flat peach teapot. On Sep. 10, 2019, the blood pressure value dropped to 80-120 mmHg.

### Embodiment 13

A patient, Mr. Li, had diabetes. On Jul. 23, 2019, his blood sugar value was 14 mmol/L, which is diagnosed as hyperglycemia, and he did not take medicine. He drunk a water of 2000 mL˜3000 mL with a concentration of lithium ions of 0.07˜4.99 mgmg/L per day. The water with the lithium ions was prepared by boiling a mineral water in a flat peach teapot. On Friday, Aug. 23, 2019, the blood pressure value dropped to 6 mmol/L.

In summary, through the above embodiments, it can be seen that when the patients suffering from hyperuricemia, gout, hypertension and hyperglycemia use the aqueous solution with the concentration of lithium ions of 0.07 mg/L to 4.99 mg/L in the invention, the uric acid value in human body may be significantly reduced; the aqueous lithium ion solution with a concentration of 0.07 mg/L to 4.99 mg/L in the invention has the effect of improving or treating hyperuricemia, gout, hypertension and hyperglycemia.

The embodiments of the invention described above do not constitute a limitation on the scope of protection of the invention. Any modification, equivalent replacement, improvement and the like made within the spirit and principle of the invention should be included in a scope of protection of the claims of the invention.

1 [symbol] A lithium ion composition, comprising lithium ions as an active substance, wherein a concentration of the lithium ions is 0.07 mg/L˜4.99 mg/L.

2 [symbol] The lithium ion composition according to claim 1, wherein the lithium ions serve as the only active substance.

3 [symbol] The lithium ion composition according to claim 1, wherein the lithium ion composition is an aqueous lithium ion solution.

4 [symbol] The lithium ion composition according to claim 3, wherein the aqueous lithium ion solution is prepared by boiling an ordinary water in a flat peach teapot.

5 [symbol] The lithium ion composition according to claim 4, wherein the ordinary water is one or more of a tap water, a mineral water and a pure water.

6 [symbol] An application of the lithium ion composition according to claim 1 in products for treating hyperuricemia, gout, hypertension and hyperglycemia.

7 [symbol] The application of the lithium ion composition in products for treating hyperuricemia, gout, hypertension and hyperglycemia according to claim 6, wherein the product is any one of a drug and a food.
